(19) United States
(12) Patent Application Publication
(10) Pub. No.: US 2020/0319164 A1
(43) Pub. Date: Oct. 8, 2020

(54) METHODS FOR DETECTING, ASSESSING SEVERITY AND TREATING MULTIPLE SCLEROSIS

(71) Applicants: INSERM (INSTITUT NATIONAL DE LA SANTE ET DE LA RECHERCHE MEDICALE), paris (FR); UNIVERSITÉ DE NANTES, Nantes (FR); CENTRE HOSPITALIER UNIVERSITAIRE DE NANTES, Nantes (FR)

(72) Inventors: Carole GUILLONNEAU, Nantes (FR); David LAPLAUD, NANTES CEDEX 1 (FR); Séverine BEZIE, Nantes (FR); Bryan NICOL, Nantes (FR)

(21) Appl. No.: 16/303,771

(22) PCT Filed: May 24, 2017
(86) PCT No.: PCT/EP2017/062500

§ 371 Date: Nov. 21, 2018

(30) Foreign Application Priority Data
May 25, 2016 (EP) 16305606.2

(51) Int. Cl. G01N 33/50; G01N 33/68

(52) U.S. Cl. CPC G01N 33/5091; G01N 33/5094; G01N 33/6893; G01N 2333/5428; G01N 2800/56; G01N 2800/50; G01N 2333/57; G01N 2800/285

(57) ABSTRACT

The present invention relates to methods for detecting, assessing severity and treating multiple sclerosis. The inventors showed an impairment of the function of CD8+ Treg cells in MS patients and they demonstrated here that several criteria correlated with the disease severity i.e. the percentage of CD8⁺CD45RC^{int}CD161^{low}Valpha7⁻ T cells in the blood, the secretion of IFNg and IL10 and the suppressive activity of the CD8⁺CD45RC^{int}CD161^{low}Valpha7⁻ T cells. In particular, the present invention relates to a method for determining whether a subject has or is at risk of having multiple sclerosis comprising i) determining the percentage of CD8⁺CD45RC^{int}CD161^{low}Valpha7⁻ T cells in a biological sample obtained from the subject, ii) comparing the percentage determined at step i) with a predetermined reference value and iii) detecting differential in the percentage determined at step i) with the predetermined reference value indicates that the subject has or is at risk of having multiple sclerosis.

### FIELD OF THE INVENTION

The present invention relates to methods for detecting, assessing severity and treating multiple sclerosis.

### BACKGROUND OF THE INVENTION

Multiple sclerosis (MS) is a chronic inflammatory and demyelinating disease of the Central Nervous System (CNS), probably of autoimmune origin. Autoimmune diseases can develop following pathological activation of autoreactive effector cells and/or, alternatively, after weakening of self-protective regulatory mechanisms, for example regulatory T cells (Tregs). While most of the studies about the role of Tregs in autoimmunity have focused on CD4⁺ T cells, the role of CD8⁺ Tregs in MS remains largely unexplored. It has been shown that some subsets of CD8⁺ T cells, including CD8⁺ Qa-1⁺, CD8⁺CD28⁻ and CD8⁺CD122⁺ T cells, can have a regulatory role in Experimental Autoimmune Encephalomyelitis (EAE), the mouse model of MS (Hu D et al., 2004; Najafian N et al., 2003; Yu P et al., 2014). However, in human, CD8⁺ Tregs are less described. Recently, the inventors have described a new subset of regulatory CD8⁺CD45RC^{low} Treg subpopulation, able to produce IFNγIL10 and IL34 and displaying a great efficiency in regulating Graft vs Host Disease (GVHD) and human skin transplantation rejection in humanized mice (Picarda et al, JCI, 2014 and Bezie et al, JCI, 2015, Bézie et al., in preparation). However the role of said population in the onset and progression of multiple sclerosis has never been investigated.

### SUMMARY OF THE INVENTION

The present invention relates to methods for detecting, assessing severity and treating multiple sclerosis. In particular, the present invention is defined by the claims.

### DETAILED DESCRIPTION OF THE INVENTION

The inventors showed an impairment of the function of CD8+ Treg cells in MS patients and they demonstrated here that several criteria correlated with the disease severity i.e. the percentage of CD8⁺CD45RC^{int}CD161^{low}Valpha7⁻ T cells in the blood, the secretion of IFNg and IL10 and the suppressive activity of the CD8⁺CD45RC^{int}CD161^{low}Valpha7⁻ T cells. These results suggest that CD8+CD45RClow T cells and its subsets may be potentials therapeutics and prognostic tools in MS patients, correlating with the progression of the disease.

A correlation between the function of CD8⁺CD45RC^{int}CD161^{low}Valpha7⁻ T cells in the blood and the severity of the MS pathology has never been suggested or suspected. The presence of CD8+ Tregs in mice and rat has been described to protect upon transfer in autoimmune uveitis in rat and EAE in mice and thus they are functional (Sinha, S., et al. 2014. Immune regulation of multiple sclerosis by CD8+ T cells. Immunol Res 59:254-265) (Sinha, S., et al. 2015. CD8(+) T-Cells as Immune Regulators of Multiple Sclerosis. Front Immunol 6:619) (Han, G., et al. 2007. Suppressor role of rat CD8+CD45RClow T cells in experimental autoimmune uveitis (EAU). J Neuroimmunol 183:81-88). In the opposite, we describe that they are not functional, they do not protect and the more the MS patient is sick, the more dysfunctional they are, in contrast to previous articles.

As used herein the term “multiple sclerosis” or “MS” has its general meaning in the art and is used to describe the art-recognized disease characterized by inflammation, demyelination, oligodendrocyte death, membrane damage and axonal death. MS can be more particularly categorized as either relapsing/remitting MS (observed in 85-90% of patients) or progressive MS. In some embodiments, MS can be characterized as one of four main varieties as defined in an international survey of neurologists (Lublin and Reingold, 1996, Neurology 46(4):907-11), which are namely, relapsing/remitting MS, secondary progressive MS, progressive/relapsing MS, or primary progressive MS (PPMS).

Accordingly, the first object of the present invention relates to a method for determining whether a subject has or is at risk of having multiple sclerosis comprising i) determining the percentage of CD8⁺CD45RC^{int}CD161^{low}Valpha7⁻T cells in a biological sample obtained from the subject, ii) comparing the percentage determined at step i) with a predetermined reference value and iii) detecting differential in the percentage determined at step i) with the predetermined reference value indicates that the subject has or is at risk of having multiple sclerosis.

The second object of the present invention relates to a method for assessing or predicting the severity of multiple sclerosis in a subject comprising i) determining the percentage of CD8⁺CD45RC^{int}CD161^{low}Valpha7⁻ T cells in a biological sample obtained from the subject, ii) comparing the percentage determined at step i) with a predetermined reference value and iii) detecting differential in the percentage determined at step i) with the predetermined reference value indicates the severity of the disease.

The third object of the present invention relates to a method for determining whether a subject has or is at risk of having multiple sclerosis comprising i) determining the production level of at least one cytokine by the population of CD8⁺CD45RC^{int}CD161^{low}Valpha7⁻ T cells in a biological sample obtained from the subject, ii) comparing the production level determined at step i) with a predetermined reference value and iii) detecting differential in the production level determined at step i) with the predetermined reference value indicates that the subject has or is at risk of having multiple sclerosis.

The fourth object of the present invention relates to a method for assessing or predicting the severity of multiple sclerosis in a subject comprising i) determining the production level of at least one cytokine by the population of CD8⁺CD45RC^{int}CD161^{low}Valpha7⁻ T cells in a biological sample obtained from the subject, ii) comparing the production level determined at step i) with a predetermined reference value and iii) detecting differential in the production level determined at step i) with the predetermined reference value indicates the severity of the disease.

In some embodiments, the cytokine is selected from the group consisting of IL-34, IFNγ, IL-2 and IL-10. In some embodiments, the method of the present invention comprises determining the production level of IFNγ. In some embodiments, the method of the present invention comprises determining the production level of IFNγ and IL-10.

The fifth object of the present invention relates to a method for determining whether a subject has or is at risk of having multiple sclerosis comprising i) assessing the suppressive activity of the population of CD8⁺CD45RC^{int}CD161^{low}Valpha7⁻T cells present in a biological sample obtained from the subject, ii) comparing the suppressive activity determined at step i) with a predetermined reference value and iii) detecting differential in the suppressive activity determined at step i) with the predetermined reference value indicates that the subject has or is at risk of having multiple sclerosis.

The sixth object of the present invention relates to a method for assessing or predicting the severity of multiple sclerosis in a subject comprising i) assessing the suppressive activity of the population of CD8⁺CD45RC^{int}CD161^{low}Valpha7⁻ T cells present in a biological sample obtained from the subject, ii) comparing the suppressive activity determined at step i) with a predetermined reference value and iii) detecting differential in the suppressive activity determined at step i) with the predetermined reference value indicates the severity of the disease.

As used herein, the term “risk” refers to the probability that an event will occur over a specific time period, such as the onset of multiple sclerosis, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a patient compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event). “Risk determination” in the context of the invention encompasses making a prediction of the probability, odds, or likelihood that an event may occur. Risk determination can also comprise prediction of future clinical parameters, traditional laboratory risk factor values, such age, sex mismatch, HLA-testing, etc. either in absolute or relative terms in reference to a previously measured population. The methods of the invention may be used to make categorical measurements of the risk of multiple sclerosis, thus defining the risk spectrum of a category of transplanted patient defined as being at risk of multiple sclerosis.

In some embodiments, the percentage of the population of interest, the production level of the cytokine and the suppressive activity correlates with the severity. “Severity” of Multiple Sclerosis (MS) may be expressed according to the invention with any means known in the field of MS like e.g. Expanded Disease Status Scale (EDSS), MSSS score (multiple sclerosis severity score) with other commonly used techniques or measurements or definitions in the field. Examples of the assessment or diagnosis of MS are published in Kurzke J. F., Neuroepidemiology, 1991, 10: 1-8; Kurzke J. F., Neurology, 1983, 33: 1444-1452; McDonald W. I et ai, Ann. Neurol., 2001, 50: 121-127; Polman CH. et al., Ann. Neurol. 2005, 58: 840-846. Accordingly, a severity marker or SNP may represent a marker indicating high disease or low disease severity in a patient as compared to the MS population.

As used herein, the term “regulatory T cells” or “Tregs”, formerly known as suppressor T cells, refers to a subpopulation of T cells which modulate the immune system, maintain tolerance to self-antigens, and abrogate autoimmune disease. These cells generally suppress or downregulate induction and proliferation of effector T cells.

As used herein the term “population of CD8⁺CD45RC^{int}CD161^{low}Valpha7⁻ T cells” refers to a subset of Treg cells characterized by the expression of CD8, an intermediate or low expression of CD45RC,by the absence or low expression of CD161 and by the absence of expression of Valpha7. The population is also characterized by the production of IL-34, IL-2, IL-10 and IFNγ. See FIG. 7 for the understanding of CD8⁺CD45RC^{int}CD161^{low}Valpha7⁻ T cells characterization.

As used herein, the term “CD8” (cluster of differentiation 8) well known in the art refers to a transmembrane glycoprotein that serves as a co-receptor for the T cell receptor (TCR). To function, CD8 forms a dimer, consisting of a pair of CD8 chains. The naturally occurring human CD8-α protein has an aminoacid sequence provided in the UniProt database under accession number P01732. The naturally occurring human CD8-β protein has an aminoacid sequence provided in the UniProt database under accession number P10966.

As used herein, the term “CD45” (also known as LCA or PTPRC) refers to a transmembrane glycoprotein existing in different isoforms previously described in Streuli et al., 1996. These distinct isoforms of CD45 differ in their extracellular domain structures which arise from alternative splicing of 3 variable exons coding for part of the CD45 extracellular region. The various isoforms of CD45 have different extracellular domains, but have the same transmembrane and cytoplasmic segments having two homologous, highly conserved phosphatase domains of approximately 300 residues. The naturally occurring human CD45 protein has an aminoacid sequence provided in the UniProt database under accession number P08575. As used herein, the term “CD45RC” refers to the exon 6 splice variant (exon C) of the tyrosine phosphatase CD45. The CD45RC isoform is expressed on B and T cells.

As used herein the term “CD161” has its general meaning in the art and refers to killer cell lectin like receptor B1 (Gene ID:3820). CD161 is also known as NKR; CLEC5B; NKR-P1; NKRP1A; NKR-P1A; and hNKR-P1A. An exemplary amino acid sequence for CD161 is provided by the NCBI reference sequence NP_002249.1.

As used herein the term “Valpha7”, also known as TRAV1-2, has its general meaning in the art and refers to the T cell receptor V alpha 7 chain (Homo sapiens NCBI gene ID: 28692).

The terms “low” and “int” are general terms of the skilled person in cytometry for qualifying the expression level of a surface marker. In particular, the term “low” indicates that the surface marker is expressed at an intermediate level “int” or is null.

As used herein, the terms “Interferon gamma” or “IFNγ”, or “type II interferon” are well known in the art and refer to a cytokine that is critical for innate and adaptive immunity. The naturally occurring human IFNγ protein has an aminoacid sequence of 146 amino acids provided in the UniProt database under accession number P01579.

As used herein the terms “Interleukin-2” or “IL-2” are well known in the art and refer to cytokine which is important for the proliferation of T and B lymphocytes. The naturally occurring human IL-2 protein has an aminoacid sequence of 133 amino acids provided in the UniProt database under accession number P60568.

As used herein, the terms “Interleukin-10” or “IL10” are well known in the art and refer to an anti-inflammatory cytokine. The naturally occurring human IL-10 protein has an aminoacid sequence of 178 amino acids provided in the UniProt database under accession number P22301.

As used herein, the terms “Interleukin-34” or “IL34” are well known in the art and refer to a cytokine that promotes the proliferation, survival and differentiation of monocytes and macrophages. The naturally occurring human IL-34 protein has an aminoacid sequence of 242 amino acids provided in the UniProt database under accession number Q6ZMJ4.

As used herein the term “biological sample” refers to any sample obtained from the subject liable to contain T cells. In some embodiments, the biological sample is a blood sample. In some embodiments, the biological sample is a cerebrospinal fluid sample. In some embodiments, the biological sample is a biopsy sample. In some embodiments, the biological sample is a PBMC sample. The term “PBMC” or “peripheral blood mononuclear cells” or “unfractionated PBMC”, as used herein, refers to whole PBMC, i.e. to a population of white blood cells having a round nucleus, which has not been enriched for a given sub-population. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art. The template nucleic acid need not be purified. Nucleic acids may be extracted from a sample by routine techniques such as those described in Diagnostic Molecular Microbiology: Principles and Applications (Persing et al. (eds), 1993, American Society for Microbiology, Washington D.C.).

The quantification and isolation of the population of the CD8⁺CD45RC^{int}CD161^{low}Valpha7⁻ T cells may be carried out by a variety of methods for detecting a particular immune cell population available for a skilled artisan, including immunoselection techniques, such as high-throughput cell sorting using flow cytometric methods, affinity methods with antibodies labeled to magnetic beads, biodegradable beads, non-biodegradable beads, and combination of such methods. As used herein, the term “flow cytometric methods” refers to a technique for counting cells of interest, by suspending them in a stream of fluid and passing them through an electronic detection apparatus. Flow cytometric methods allow simultaneous multiparametric analysis of the physical and/or chemical parameters of up to thousands of particles per second, such as fluorescent parameters. Modern flow cytometric instruments usually have multiple lasers and fluorescence detectors. A common variation of flow cytometric techniques is to physically sort particles based on their properties, so as to purify or detect populations of interest, using “fluorescence-activated cell sorting”. As used herein, “fluorescence-activated cell sorting” (FACS) refers to a flow cytometric method for sorting a heterogeneous mixture of cells from a biological sample into two or more containers, one cell at a time, based upon the specific light scattering and fluorescent characteristics of each cell and provides fast, objective and quantitative recording of fluorescent signals from individual cells as well as physical separation of cells of particular interest. Accordingly, FACS can be used with the methods described herein to isolate for instance human CD8⁺ Treg cells. Alternatively, isolation of the population of interest can be performed using bead based sorting methods, such as magnetic beads. Using such methods, cells can be separated and isolated positively or negatively with respect to the particular cell-surface markers. As defined herein, “positive selection” refers to techniques that result in the isolation and detection of cells expressing specific cell-surface markers, while “negative selection” refers techniques that result in the isolation and detection of cells not expressing specific cell-surface markers. In some embodiments, beads can be coated with antibodies by a skilled artisan using standard techniques known in the art, such as commercial bead conjugation kits. In some embodiments, a negative selection step is performed to remove cells expressing one or more lineage markers, followed by fluorescence activated cell sorting to positively select human Treg cells of interest.

In some embodiments, the biological sample is contacted with a panel of binding partners (e.g. antibodies) having specificity of CD3, CD8/4, CD45RC, CD161 and Valpha7 and positive and negative selection can be then performed for isolating and quantifying the population of interest.

In some embodiments, the methods involve those described in EP 15 305 715.3. In particular, means useful for isolating the population of interest are binding partners (such as antibodies) to suitable cell surface molecules or markers. Specific binding partners include capture moieties and label moieties. The capture moieties are those which attach both to the cell, either directly or indirectly, and the product. The label moieties are those which attach to the product and may be directly or indirectly labeled. Specific binding partners include any moiety for which there is a relatively high affinity and specificity between product and its binding partner, and in which the dissociation of the product: partner complex is relatively slow so that the product: partner complex is detected during the labeling or cell separation technique. The capture moiety may be coupled to the anchoring means (the “anchor moiety”) optionally through a linking moiety, and may also include a linking moiety which multiplies the number of capture moieties available and thus the potential for capture of product, such as branched polymers, including, for example, modified dextran molecules, polyethylene glycol, polypropylene glycol, polyvinyl alcohol, and polyvinylpyrrolidone. When the capture moiety is an antibody it may be referred to as the “capture antibody” or “catch antibody.” As used herein, the term “antibody is intended to include polyclonal and monoclonal antibodies, chimeric antibodies, single domains antibodies, haptens and antibody fragments, bispecific antibodies, trispecific antibodies and molecules which are antibody equivalents in that they specifically bind to an epitope on the product antigen. In one embodiment, the capture moiety is selected from the group consisting of a bispecific antibody which binds to CD8 and IL34, and a bispecific antibody which binds to CD45RC and IL34. In some embodiments, the capture moiety binds to IL34 and to other cytokines such as IFNγ, IL10 and IL-34. In some embodiments the capture moiety is a trispecific antibody which binds to IL34, IFNγ and CD8 or a trispecific antibody which binds to IL34, IFNγ and CD45RC.

Typically, the antibodies are labeled. The label moiety that can be conjugated to a binding partner such as an antibody are well known to the skilled in the art. For example, radioisotopes, e.g. ³²P, ³⁵S or ³H; fluorescence or luminescence markers, e.g. fluorescein (FITC), rhodamine, texas red, phycoerythrin (PE), allophycocyanin, peridinin-chlorophyll-protein complex (PerCP), 6-carboxyfluorescein (6-FAM), 2′, 7′-dimethoxy-4′, 5′-dichloro-6-carboxyfluorescein (JOE), 6-carboxy-X-rhodamine (ROX), 6-carboxy-2′, 4′, 7′, 4, 7-hexachlorofluorescein (HEX), 5-carboxyfluorescein (5-FAM) or N, N, N′, N′-tetramethyl-6-carboxyrhodamine (TAMRA); antibodies or antibody fragments, e.g. F(ab)2 fragment; affinity labels, e.g. biotin, avidin, agarose, bone morphogenetic protein (BMP), matrix bound, haptens; and enzymes or enzyme substrates, e.g. alkaline phosphatase (AP) and horseradish peroxidase (HRP).

The production of the cytokine of interest may be determined by any assay well known in the art. In some embodiments, said assay involved flow cytometry as described in the EXAMPLE. In some embodiments, said assay may consist in an enzyme-linked immunospot (ELISpot) assay. Non-adherent cells from pre-culture wells are transferred to a plate which has been coated with the desired anti-cytokine capture antibodies (Abs; e.g., anti-IFN-γ, -IL-10, -IL-2). Revelation is carried out with biotinylated secondary Abs and standard colorimetric or fluorimetric detection methods such as streptavidin-alkaline phosphatase and NBT-BCIP and the spots counted. In some embodiments, the assay may consist in a cytokine capture assay. This system developed by Miltenyi Biotech is a valid alternative to the ELISPOT to visualize antigen-specific T cells according to their cytokine response. In addition, it allows the direct sorting and cloning of the population of interest. In some embodiments, the assay may consist in a supernatant cytokine assay. Cytokines released in the culture supernatant are measured by different techniques, such as enzyme-linked immunosorbent assays (ELISA), BD cytometric bead array, Biorad or Millipore cytokine mutiplex assays and others.

Methods for determining the suppressive activity of the population of interest are also well known in the art and typically include the assay described in the EXAMPLE. In particular, the suppressive activity was assessed on syngeneic effector CD4+CD25−T cells stimulated with allogeneic APCs. Then, the proliferation of the CD4+CD25−T cells are determined. The method may consist in a CFSE dilution assay. This procedure detects T cells according to their proliferation following antigenic recognition [Mannering et al., J. Immunol. Methods 283:173, 2003].

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the biomarker in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the biomarker in a group of reference, one can use algorithmic analysis for the statistic treatment of the measured expression levels of the gene(s) in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

Typically, the percentage of the population of CD8⁺CD45RC^{int}CD161^{low}Valpha7⁻ T cells is deemed to be lower than the predetermined reference value established in healthy population. The lower is the percentage of the population of CD8⁺CD45RC^{int}CD161^{low}Valpha7⁻ T cells, the higher is the severity of the disease.

Typically, the production level of the cytokine is deemed to be lower than the predetermined reference value established in healthy population. The higher is the production level of IFNg, the higher is the severity of the disease.

Typically, the lower is the suppressive activity of the population of CD8⁺CD45RC^{int}CD161^{low}Valpha7⁻ T cells, the higher is the severity of the disease.

Once it is concluded that the subject suffers from multiple sclerosis and depending on the severity of the disease, he can be administered with a therapeutic treatment. The method of the present invention is thus particularly suitable for stratifying the patients and selecting the most accurate regimen of treatment. A further aspect of the present invention relates to a method of treatment of multiple sclerosis in a patient in need thereof comprising determining the severity of the disease by the method of the present invention and administering the patient with a suitable treatment. Suitable treatment include, but are not limited to, interferon, interferon beta Ia, interferon beta Ib, natalizumab (Tysabri), rixtuximab (Rituxan, MabThera), glatiramer acetate, mitoxantrone, azathioprine, cyclophosphamide, cyclosporine, dimethyl fumarate, methotrexate, cladribine, methylprednisolone, prednisone, prednisolone, dexamethasone, adreno-corticotrophic hormone, corticotrophin, carbamazepine, gabapentin, tropirmate, zonisamide, phenytoin, desipramine, amitriptyline, imipramine, doexepin, protriptyline, pentoxifylline, 4-aminopyridine, 3,4 diaminopyridine, eliprodil, pregabalin and ziconotide, ofatumumab, ocrelizumab, alemtuzumab (Campath), daclizumab (Zenapax), belimumab (Lympho-Stat-B), glatiramer acetate (Copaxone), mitoxantrone (Novantrone), azathioprine, cyclosporine, methotrexate, cyclophosphamide, intravenous immunoglobulin, prednisone, methylprednisone, prednisolone, methylprednisolone, dexamethasone, adreno-corticotrophic hormone (ACTH), corticotropin, 2-chlorodexyadenosine (2-CDA, cladribine), inosine, Interleukin-2 antibody (Zenapax, daclizumab), IL-34 polypeptide, anti-CD45RC antibody, leucovorin, teriflunomide, estroprogestins, desogestrel, etinilestradiol, BHT-3009, ABT-874, Bacille Calmette-Guerin (BCG) Vaccine, T cell vaccination, CNTO 1275, N-acetylcysteine, minocycline, RO0506997, or a statin. In some embodiments, the treatment consists in the administration of at least one corticosteroid. Corticosteroids, such as oral prednisone and intravenous methylprednisolone, are prescribed to reduce nerve inflammation. In some embodiments, the treatment consists in a plasma exchange (plasmapheresis). In some embodiments, the treatment consists in the administration of beta interferons, glatiramer acetate (Copaxone), fimethyl fumarate (Tecfidera),fingolimod (Gilenya), teriflunomide (Aubagio), natalizumab (Tysabri), Alemtuzumab (Lemtrada), Mitoxantrone and Laquinimod sodium. In some embodiments, the subject is administered with a therapeutically effective amount of a population of Treg cells. In some embodiments, the T regulatory (Treg) cells may be genetically modified to encode desired expression products, as will be further described below. A number of approaches can be used to genetically modify Treg cells, such as virus-mediated gene delivery, non-virus-mediated gene delivery, naked DNA, physical treatments, etc. To this end, the nucleic acid is usually incorporated into a vector, such as a recombinant virus, a plasmid, phage, episome, artificial chromosome, etc. In some embodiments, the nucleic acid encodes a T cell receptor or a sub-unit or functional equivalent thereof such as a chimeric antigen receptor (CAR) specific to an antigen of interest. For instance, the expression of recombinant TCRs or CAR specific for an antigen produces Treg cells which can act more specifically and efficiently on effector T cells to inhibit immune responses in a patient in need thereof. The basic principles of chimeric antigen receptor (CAR) design has been extensively described (e.g. Sadelain et al., 2013). The CAR may be first generation, second generation, or third generation (CAR in which signaling is provided by CD3 together with co-stimulation provided by CD28 and a tumor necrosis factor receptor (TNFr), such as OX40), for example. CAR are obtained by fusing the extracellular antigen-binding domain of the mAb with the intracellular signaling domains derived from the CD3-ζ chain of the T-cell receptor, in tandem to costimulatory endodomains to support survival and proliferative signals. Because CAR-modified T cells function independently of a patient's MHC and can readily be generated for clinical use, the targeting of pathogenic antigen as described below with a CAR based-approach is useful.

The invention will be further illustrated by the following figures and examples. However, these examples and figures should not be interpreted in any way as limiting the scope of the present invention.

### FIGURES

FIG. 1: Percentage of peripheral human CD8⁺CD45RC^{int} Tregs subsets in MS patients. CD8⁺CD45RC^{int} T cells were analyzed in the blood of MS patients vs. healthy individuals for expression level of CD45RC.

FIG. 2: Percentage of peripheral blood and brain infiltrating human CD8⁺CD45RC^{int} Tregs subsets in MS patients. CD8⁺CD45RC^{int} T cells were analyzed in the blood and cerebrospinal fluids of MS patients for expression level of CD45RC.

FIG. 3: Percentage of human CD8⁺CD45RC^{low} Tregs expressing IL-2, IFNg and/or IL10. CD8⁺CD45RC^{low} T cells (discriminating CD45RC^{int} and CD45RC^{neg}) were analyzed in the blood of MS patients vs. healthy individuals for expression level of IL-2, IFNg and/or IL10.

FIG. 4: Percentage of CD8⁺CD45RC^{int} Tregs expressing IFNg in MS patients with low or high MSSS. CD8⁺CD45RC^{int} T cells were analyzed in the blood of MS patients for expression level of IFNg.

FIG. 5. Dose dependent inhibition of the proliferation of effector CD4⁺CD25⁻ T cells by CD8⁺CD45RC^{int}. CFSE labelled recipient CD4⁺CD25⁻ effector T cells were mixed with allogeneic T-depleted PBMCs at a 1:1 ratio and CD3⁺CD161lowValpha7−CD8⁺CD45RC^{int} Tregs were added at different ratios. Proliferation of CD4⁺CD25⁻ T cells was analysed 5 days later by flow cytometry. upper panel represents suppressive capacity in MS patients with a high MSSS number (>3) and lower panel represents suppressive capacity of MS patients with a low MSSS number (<3) compared to healthy individuals. * p<0.05; ** p<0.01; Two way anova RM, Bonferroni post test.

FIG. 6. Dose dependent inhibition of the proliferation of effector CD4⁺CD25⁻ T cells by CD8⁺CD45RC^{int}. CFSE labelled recipient CD4⁺CD25⁻ effector T cells were mixed with allogeneic T-depleted PBMCs at a 1:1 ratio and CD3+CD161lowValpha7-CD8⁺CD45RC^{low/neg/int} Tregs were added at different ratios. Proliferation of CD4⁺CD25⁻ T cells was analysed 5 days later by flow cytometry. upper panel represent suppressive capacity in MS patients with a high MSSS number (>3) compared with MS patients with a low MSSS number (<3). lower panel represent suppressive capacity in function of MSSS number at effector: stimulator: suppressor ratio 4:4:1 (left) and 2:2:1 (right). Two Way RM ANOVA, Bonferroni post test, * p<0.05; ** p<0.01;*** p<0.001.

FIG. 7. Gating strategy to identify CD8⁺CD45RClowCD161-Va7⁻ Tregs.

### EXAMPLE 1

Material & Methods

Healthy volunteers blood collection and PBMC separation: Blood was collected from healthy donors at the Etablissement Francais du Sang (Nantes, France). Approval for this study was obtained from the institutional review boards. Written informed consent was provided according to institutional guidelines. Blood was diluted 2-fold with PBS before PBMC were isolated by Ficoll-Paque density-gradient centrifugation (Eurobio, Courtaboeuf, 10 France) at 2000 rpm for 20 at room temperature without braking. Collected PBMC were washed in 50 mL PBS at 1800 rpm for 10 min and remaining red cells and platelets are eliminated after incubation 5 min in a hypotonic solution and centrifugation at 1000 rpm for 10 min. When indicated, PBMCs were frozen in DMSO:SVF 1:9 and washed twice in medium-10% SVF for thawing.

Cell Isolation:

T cells were obtained from PBMCs by negative selection by elutriation (DTC Plateforme, Nantes) and magnetic depletion (Dynabeads, Invitrogen) of B cells (CD19+ cells) and remaining monocytes (CD14+ and CD16+ cells) before sorting of CD3+CD4+CD25− cells as responder cells, CD3+CD161^{low}Valpha7⁻CD4⁻CD45RC^{low} (int or neg) as CD8+ Tregs. Tregs sorted from thawed PBMCs were stimulated 24 h with anti-CD3 and anti CD28 mAb (1 μg/ml each) in presence of 250 U/ml IL-2 before plating. A FACS ARIA I (BD Biosciences, Mountain View, Calif.) was used to sort cells. APCs used as stimulator cells were obtained by magnetic depletion of CD3+ cells and 35Gy irradiation.

Monoclonal Antibodies and Flow Cytometry:

For interleukins and IFNg analysis, PBMCs were stimulated with PMA (50 ng/ml) and ionomycine (1 μg/ml) for 7 h in presence of Brefeldine A (10 g/ml) for the last 4 h. Fluorescence was measured with a LSR II or a Canto II cytometer (BD Biosciences, Mountain View, Calif.) for phenotype and functional analysis respectively, and the FLOWJO software (Tree Star, Inc. USA) was used to analyze data. Cells were first gated by their morphology excluding dead cells by selecting viable cells.

Mixed Lymphocyte Reaction:

Tregs suppressive activity was assessed on syngeneic effector CD4+CD25−T cells stimulated with allogeneic APCs. Experiments were realized with 1:1 APCs: responder ratio. Proliferation of CFSE-labeled responder cells was analyzed by flow cytometry after 4.5 days of coculture in 5% AB serum medium, by gating on CD3+CD4+ living cells (DAPI negative).

Results

Multiple sclerosis (MS) is a chronic inflammatory and demyelinating disease of the Central Nervous System (CNS), probably of autoimmune origin. Autoimmune diseases can develop following pathological activation of autoreactive effector cells and/or, alternatively, after weakening of self-protective regulatory mechanisms, for example regulatory T cells (Tregs). While most of the studies about the role of Tregs in autoimmunity have focused on CD4⁺ T cells, the role of CD8⁺ Tregs in MS remains largely unexplored. It has been shown that some subsets of CD8⁺ T cells, including CD8⁺ Qa-1⁺, CD8⁺CD28⁻ and CD8⁺CD122⁺ T cells, can have a regulatory role in Experimental Autoimmune Encephalomyelitis (EAE), the mouse model of MS (Hu D et al., 2004; Najafian N et al., 2003; Yu P et al., 2014). However, in human, CD8⁺ Tregs are less described.

Recently, we have described a new subset of regulatory CD8⁺CD45RC^{low} Treg subpopulation, able to produce IFNγ, IL10 and IL34 and displaying a great efficiency in regulating Graft vs Host Disease (GVHD) and human skin transplantation rejection in humanized mice (Picarda et al, JCI, 2014 and Bezie et al, JCI, 2015, Bézie et al., in preparation). The aim of our study is to analyze the frequency and function of this particular subset of CD8⁺ T cells in the blood of MS patients compared to Healthy Volunteers (HV) and in cerebrospinal fluids of MS patients.

We have recruited age- and gender-matched 22 MS patients and 24 healthy volunteers. Inclusion criteria were as follows: age between 18 and 55, relapsing MS, EDSS between 0-5.5, immunomodulatory treatment stopped at least 6 months ago, immunosupressant treatment stopped at least 12 months ago, natalizumab stopped at least three months ago. Importantly, all our analysis excluded MAIT cells (CD8⁺CD161^{high}Valpha7⁺ cells) that may have a role in MS. In addition, we discriminated in all our analysis CD45RC^{high} (data not shown), CD45RC^{int} and CD45RC^{neg}.

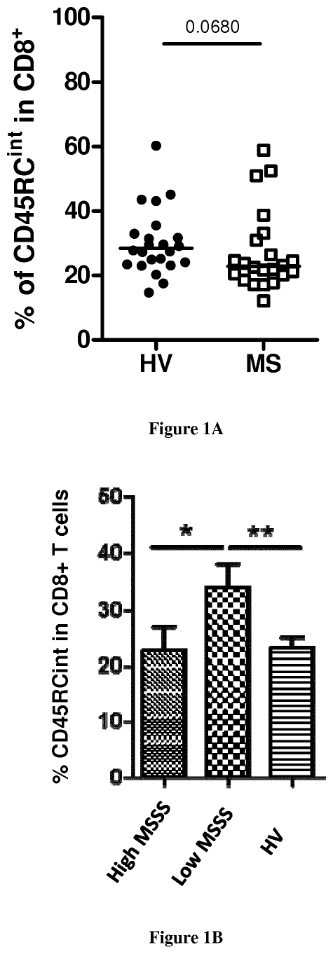

Analysis of the CD8⁺CD161^{low}Valpha7-CD3⁺CD45RC subpopulations of cells in MS patients versus healthy volunteers showed a trend for a decreased in the percentage of CD8⁺CD45RC^{int}CD161^{low}Valpha7-CD3⁺ cells and no differences for the other subsets (FIG. 1A and data not shown). Interestingly, a more precise analysis showed a significant increase of the frequency of CD8⁺CD45RC^{int}CD161^{low}Valpha7⁻CD3⁺ but not CD8⁺CD45RC^{neg}CD161^{low}Valpha7⁻CD3⁺ T or CD8⁺CD45RC^{high}CD161^{low}Valpha7⁻CD3⁺ T cells in the blood of MS patients with a low MSSS compared to HV and MS patients with a high MSSS, demonstrating that an increased frequency of CD8⁺CD45RC^{int}CD161^{low}Valpha7⁻ CD3⁺ Tregs correlated with a lower disease (FIG. 1B and data not shown).

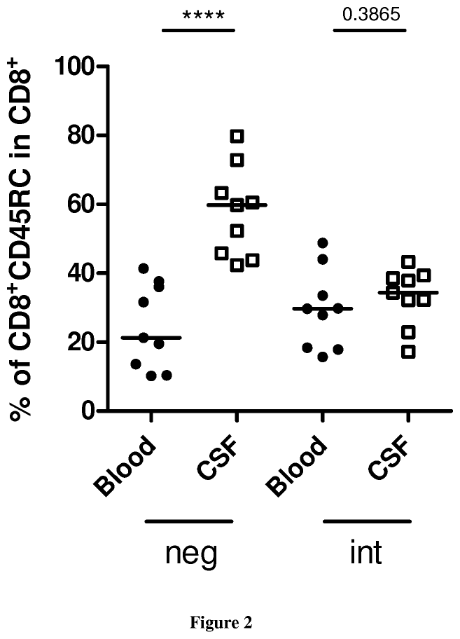

Analysis in MS patients in the blood versus the cerebrospinal fluid (CSF) demonstrated significantly more CD8⁺CD45RC^{neg}CD161^{low}Valpha7⁻ CD3⁺ T cells but similar levels of CD8⁺CD45RC^{int}CD161^{low}Valpha7⁻CD3⁺ T cells in the CSF of MS patients compared to the blood (FIG. 2).

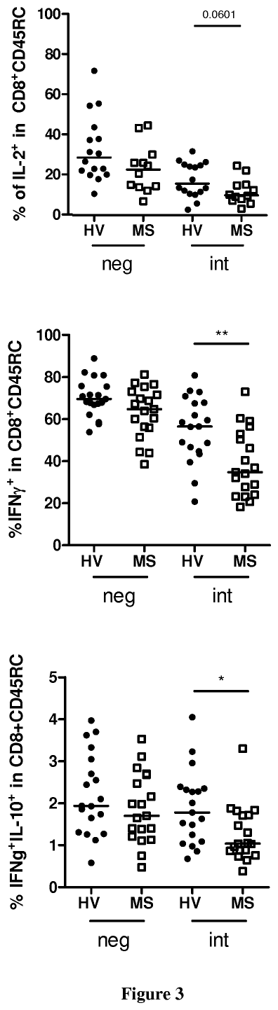

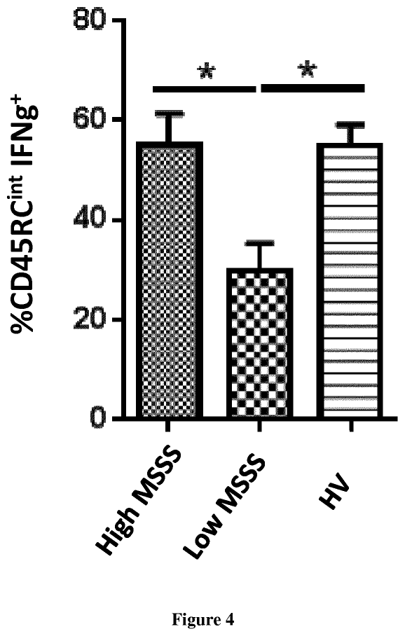

Analysis of cytokine secretion capacity upon short-term stimulation demonstrated that a significant decrease in production of IFNγ, IL-2 and IL-10/IFNγ by CD8⁺CD45RC^{int}CD161^{low}Valpha7⁻CD3⁺ T cells from MS patients compared to controls (FIG. 3). Further analysis revealed that the altered IFNg secretion profile in CD8⁺CD45RC^{int}CD161^{low}Valpha7⁻CD3⁺ T cells in the blood of MS patients correlated with a low severity score versus healthy volunteers and MS patients with a high severity score (FIG. 4).

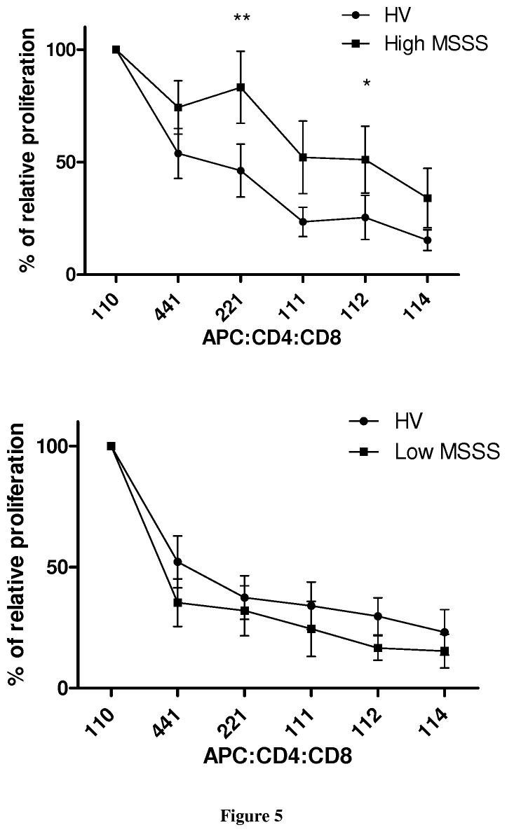

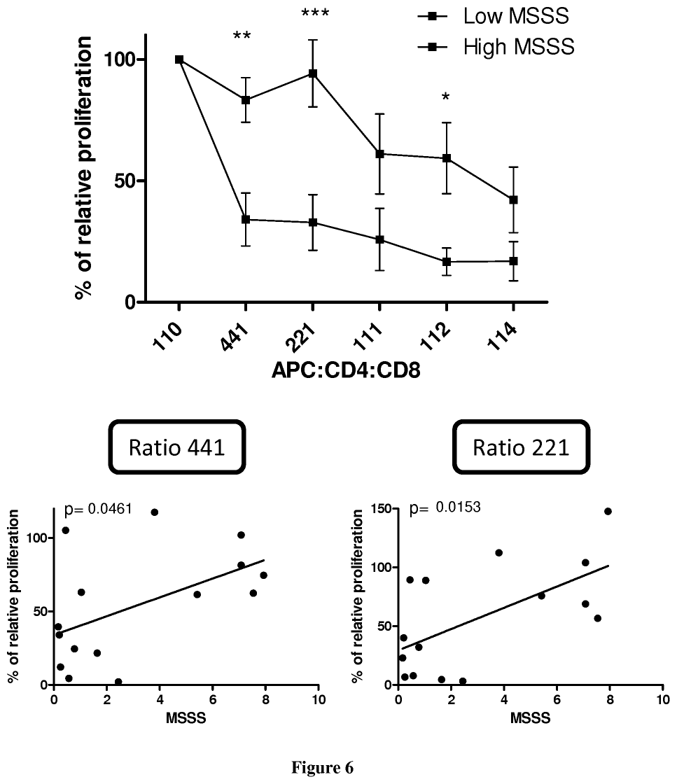

Next, CD8⁺CD45RC^{int, neg or high} CD161^{low}Valpha7⁻CD3⁺T cells from MS or HV individuals were evaluated for their suppressive activity on proliferation of syngeneic effector CD4⁺CD25⁻T cells stimulated with allogeneic T-depleted PBMCs, in an effector: suppressor dose dependent manner (FIG. 5). We observed that the suppressive capacity of CD8⁺CD45RC^{int} CD161^{low}Valpha7⁻CD3⁺T cells from high MSSS patients was significantly altered while the one from low MSSS patients remained unchanged and similar to HV. In addition, we demonstrated that the suppressive activity of CD8⁺CD45RC^{int}CD161⁻ T cells from MS patients correlated with the severity of the MS disease (FIG. 6).

Altogether, these results suggest an impairment of the function of CD8⁺ Treg cells in MS patients and we demonstrated here that several criteria correlated with the disease severity i.e. the percentage of CD8⁺CD45RC^{int}CD161⁻ T cells in the blood, the secretion of IFNg and the suppressive activity of the CD8⁺CD45RC^{int}CD161⁻ T cells.

This study suggests that CD8⁺CD45RC^{low} and its subsets may be potential therapeutic and prognostic tools in MS patients, correlating with the progression of the disease.

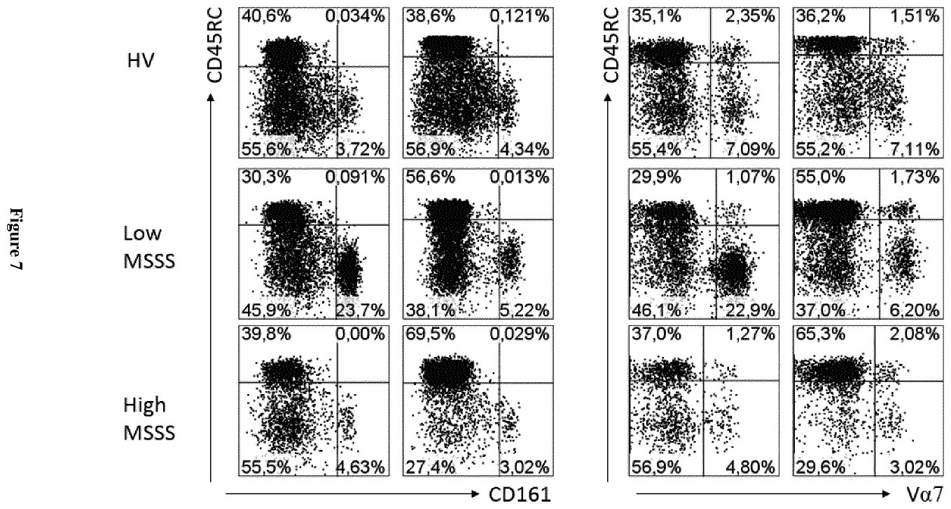

### EXAMPLE 2: MATERIAL AND METHODS OF RESULTS SHOWED IN FIG. 7

### MLR:

PBMCs from MS patients and age and sexe-matched healthy volunteers were thawed and washed in medium, counted, and cell concentration was adjusted at 2×10⁸ PBMC/ml in PBS-FCS-EDTA. Cells were incubated with anti-CD3-Pe, anti CD4-PerCPCy5.5, anti-CD25-APC Cy7, anti CD161-APC, anti Valpha7.2 PeCy7 and anti-CD45RC FITC mAbs 30′ 4° C. Cells were washed with PBS-FCS-EDTA, filtered on 60 μm tissue, labeled with Dapi and FACS Aria sorted on lymphocyte morphology, exclusion of doublet cells, and DAPI-CD3⁺CD4⁻ CD45RC^{low/int/neg} CD161^{low}Valpha7.2⁻ expression as Tregs and DAPI⁻CD3⁺CD4⁺CD25⁻ expression as Teff responder cells. After sorting, Tregs were washed in medium and Teff were labeled with CFSE and washed in medium. APCs were obtained by CD3⁺ cells depletion and 35Gy irradiation from thawed PBMCs pooled from 3 allogeneic healthy volunteers. Cells were plated at 1:1 Teff:APCs ratio and a range of Teff:Tregs ratio, in RPMI 1640 medium supplemented with 5% AB serum, Penicillin (100 U/ml), Streptomycin (0.1 mg/ml), Sodium pyruvate (1 mM), Glutamine (2 mM), Hepes Buffer (1 mM), and non-essential amino acids (1×). After 5 days culture, Teff proliferation was analyzed by CFSE analysis in DAPI⁻CD4⁺CD3⁺ T cells.

### FACS Pheno:

PBMCs from MS patients and age and sexe-matched healthy volunteers were thawed and washed in medium. Cells were stimulated 7 h with PMA-ionomycin in presence of Brefeldine A for the last 4 h, in RPMI 1640 medium supplemented with 5% AB serum, Penicillin (100 U/ml), Streptomycin (0.1 mg/ml), Sodium pyruvate (1 mM), Glutamine (2 mM), Hepes Buffer (1 mM), and non-essential amino acids (1X). Cells were washed in PBS, dead cells were labeled with fixable viability dye yellow 30′ 4° C. Cells were washed in PBS-FCS-EDTA, and stained with anti-CD161, anti-Valpha7.2, anti-CD8 and anti-CD45RC mAbs for 30′ 4° C. Cells were washed twice in PBS-FCS-EDTA, and permeabilized with Fixation-permeabilization kit (Ebiosciences) 30′ 4° C. Cells were washed in permeabilization buffer (Ebiosciences) and stained with anti-IFNg, anti-IL-34, and anti-IL-10 mAbs for 30′ RT. Cells were washed twice in permeabilization buffer, once in PBS-FCS-EDTA and fixed in PBS-PFA2% 20′ 4° C. LSR II and Flowjo were used to analyze cytokines expression in Yellow CD8⁺CD45RC^{low}CD161^{low}Valpha7.2⁻ cells.

### REFERENCES

Throughout this application, various references describe the state of the art to which this invention pertains. The disclosures of these references are hereby incorporated by reference into the present disclosure.

1. A method for determining whether a subject has or is at risk of having multiple sclerosis and/or, if the subject has multiple sclerosis, assessing whether the subject has or may have a severe form of multiple sclerosis, comprising
i) determining the percentage of CD8+CD45RC^{int}CD161^{low}Valpha7⁻ T cells in a biological sample obtained from the subject,
ii) comparing the percentage determined at step i) with a predetermined reference value,
wherein a differential between the percentage determined at step i) and the predetermined reference value indicates that the subject has or is at risk of having multiple sclerosis, or that the subject has a severe form of multiple sclerosis,
and
iii) administering a suitable treatment to a subject whose measurement is indicative of having or being at risk of having multiple sclerosis, or of having a severe form of multiple sclerosis.

2. (canceled)

3. A method for determining whether a subject has or is at risk of having multiple sclerosis and/or, if the subject has multiple sclerosis, assessing whether the subject has or may have a severe form of multiple sclerosis, comprising
i) determining the production level of at least one cytokine by the population of CD8+CD45RC^{int}CD161^{low}Valpha7⁻ T cells in a biological sample obtained from the subject,
ii) comparing the production level determined at step i) with a predetermined reference value,
wherein a differential between the production level determined at step i) and the predetermined reference value indicates that the subject has or is at risk of having multiple sclerosis, or that the subject has a severe form of multiple sclerosis,
and
iii) administering a suitable treatment to a subject whose measurement is indicative of having or being at risk of having multiple sclerosis, or of having a severe form of multiple sclerosis.

4. (canceled)

5. The method of claim 3 wherein the cytokine is selected from the group consisting of IL-34, IFNγ, IL-2 and IL-10.

6. The method of claim 5 which comprises determining the production level of IFNγ.

7. The method of claim 5 which comprises determining the production level of IFNγ and IL-10.

8. A method for determining whether a subject has or is at risk of having multiple sclerosis and/or, if the subject has multiple sclerosis, assessing whether the subject has or may have a severe form of multiple sclerosis, comprising
i) assessing the suppressive activity of the population of CD8+CD45RC^{int}CD161^{low}Valpha7⁻T cells present in a biological sample obtained from the subject,
ii) comparing the suppressive activity determined at step i) with a predetermined reference value,
wherein a differential between the suppressive activity determined at step i) and the predetermined reference value indicates that the subject has or is at risk of having multiple sclerosis, or that the subject has a severe form of multiple sclerosis,
and
iii) administering a suitable treatment to a subject whose measurement is indicative of having or being at risk of having multiple sclerosis, or of having a severe form of multiple sclerosis.

9. (canceled)

10. The method of claim 1, wherein the biological sample is a blood sample, a cerebrospinal sample or a biopsy sample.

11. The method of claim 10 wherein the biological sample is a PBMC sample.

12. The method of claim 3, wherein the biological sample is a blood sample, a cerebrospinal sample or a biopsy sample.

13. The method of claim 12 wherein the biological sample is a PBMC sample.

14. The method of claim 1, wherein a percentage that is the same or higher than the corresponding predetermined reference value indicates that the disease is not severe; and the method includes a step of administering a suitable treatment to a subject whose measurement is indicative of having multiple sclerosis that is not severe.

15. The method of claim 3, wherein a production level that is the same or higher than the corresponding predetermined reference value indicates that the disease is not severe; and the method includes a step of administering a suitable treatment to a subject whose measurement is indicative of having multiple sclerosis that is not severe.

16. The method of claim 8, wherein the biological sample is a blood sample, a cerebrospinal sample or a biopsy sample.

17. The method of claim 16 wherein the biological sample is a PBMC sample.

18. The method of claim 8, wherein a suppressive activity that is the same or higher than the corresponding predetermined reference value indicates that the disease is not severe; and the method includes a step of administering a suitable treatment to a subject whose measurement is indicative of having multiple sclerosis that is not severe.
